(19) United States
(12) Patent Application Publication
(10) Pub. No.: US 2007/0055207 A1
(43) Pub. Date: Mar. 8, 2007

(54) Garments

(76) Inventors: Feler J. Dureus, Brooklyn, NY (US); Rachele Jacques-Dureus, Brooklyn, NY (US)

Correspondence Address: KENYON & KENYON LLP, ONE BROADWAY, NEW YORK, NY, 10004, US

(21) Appl. No.: 11/208,258

(22) Filed: Aug. 19, 2005

(51) Int. Cl. A61M 5/32

(52) U.S. Cl. 604/198

(57) ABSTRACT

A multi-purpose garment has a material body with a first end, a second end, a first side edge, a second side edge, a first section of a closure attached to the second side edge and a second section of the closure attached to the second side edge. The first section of the closure arrangement is releasably connectable with the second section of the closure to form a releasable connection. The first side edge has a first portion abutting the first end, a second portion abutting the second end and a first convex section arranged between and connecting the first portion and the second portion, and the second side edge has a third portion abutting the first end, a fourth portion abutting the second end and a second convex section arranged between and connecting the third portion and the fourth portion.

### FIELD OF THE INVENTION

The present invention relates to garments, e.g., a multi-purpose garment.

### BACKGROUND INFORMATION

Outer garments, such as shawls and scarves, are worn over the body in order to provide warmth and/or protection from the elements. The garments are made of lengths such that they are wrapped around the head and neck, for example, to provide the protection needed. Such garments do not provide a multi-purpose capability.

### SUMMARY

According to an exemplary embodiment of the present invention, a multi-purpose garment includes a material body with a first end, a second end, a first side edge and a second side edge. A first section of a closure arrangement is attached to the second side edge, and a second section of the closure arrangement is attached to the second side edge. The first section of the closure arrangement is configured to combine with the second section of the closure arrangement to form a releasable connection. The first side edge has a first portion abutting the first end, a second portion abutting the second end and a convex section arranged between and connecting the first portion and the second portion. The second side edge has a third portion abutting the first end, a fourth portion abutting the second end and a convex section arranged between and connecting the third portion and the fourth portion. The multi-purpose garment may thus be worn as a scarf, as a shawl or as a wrap.

The closure arrangement may include any number of buttons, snaps, connections, etc. The convex section may have, e.g., a parabolic shape, another geometric shape, etc.

The multi-purpose garment may be reversible so that a user may select the exterior fabric he or she wishes to display. Additionally, the garment may be waterproof or may have an inner core of waterproof material and an exterior non-waterproof material surrounding the inner core.

An example embodiment of a garment has a body of material with a first distal end, a second distal end and two side portions connecting the first distal end and the second distal end. An opening is provided in the body of material offset from the first distal end so that the body of material may be worn as a scarf around the neck with the second distal end passing through the opening. The second distal end may have a rounded edge. A layer of reinforcing material may be placed within the body of material extending from the first distal end toward the second distal end at least to a location corresponding to a farthest distance of the opening to the first distal end. An exterior material support arrangement may be provided. The exterior material support arrangement may include, e.g., a strip or loop of material attached to one of the side portions so that, when worn as a scarf, the second distal end may pass through the opening and then through the aperture formed by the strip or loop to thereby secure the second distal end.

The garment may be reversible. The opening may have any desired shape, e.g., a line, circle, square, star, octagon, triangle, pentagon, hexagon, crescent, etc.

In an example embodiment of the present invention, a garment includes: a material body with a first end, a second end, a first side edge and a second side edge; a first section of a closure attached to the second side edge; and a second section of the closure attached to the second side edge, the first section of the closure releasably connectible with the second section of the closure. The first side edge includes a first portion abutting the first end, a second portion abutting the second end, and a first convex section arranged between and connecting the first portion and the second portion. The second side edge includes a third portion abutting the first end, a fourth portion abutting the second end, and a second convex section arranged between and connecting the third portion and the fourth portion.

The first section of the closure may include at least one button, e.g., five buttons. The second section of the closure may include at least one loop, e.g., five loops.

The first convex section and/or the second convex section may be parabolic.

The first portion, the second portion, the third portion and/or the fourth portion may be straight and/or concave.

A material of the body of material may be waterproof.

The body of material may include an inner core of waterproof material and an exterior of non-waterproof material surrounding the inner core.

The body of material may be sized to be selectively worn as a scarf, a shawl and a wrap.

According to an example embodiment of the present invention, a garment includes: a body of material including a first distal end, a second distal end, two opposing side portions connecting the first distal end and the second distal end, and an opening located along a longitudinal axis of the body of material extending between the first distal end and the second distal end, the opening arranged to between the first distal end and the second distal end; a layer of reinforcing material placed within the body of material extending longitudinally from the first distal end toward the second distal end at least to a location corresponding to a farthest distance of the opening from the first distal end; and an exterior material support arrangement connected at least to one side portion, the body of material wrappable around a neck of a wearer with the second distal end passed through the opening and secured by the exterior material support arrangement.

The opening may be circular or may have the shape of a line, a circle, a square, a star, an octagon, a triangle, a pentagon, a hexagon, a crescent, etc.

Example embodiments of the present invention are described in more detail below with reference to the appended Figures.

### BRIEF DESCRIPTION OF THE DRAWINGS

FIG. 1 is a view of a multi-purpose garment of an exemplary embodiment of the present invention as worn by an individual in a first position.

FIG. 2 is a top view of the multi-purpose garment.

FIG. 3 is a side view of a body of material of the multi-purpose garment

FIG. 4 is a side view of a closure ball of a first side of a closure arrangement of the multi-purpose garment.

FIG. 5 is a side view of a closure loop of a second side of a closure arrangement of the multi-purpose garment.

FIG. 6 is a front view of an individual wearing the multi-purpose garment in an alternative position.

FIG. 7 is a back view of an individual wearing the multi-purpose garment in the position illustrated in FIG. 6.

FIG. 8 is a front view of an individual wearing the multi-purpose garment in another alternative position.

FIG. 9 is a back view of an individual wearing the multi-purpose garment in the position illustrated in FIG. 8.

FIG. 10 is a front view of an individual wearing the multi-purpose garment in another alternative position.

FIG. 11 is a top view of a garment according to an example embodiment of the present invention.

FIG. 12 is a front view of an individual wearing the garment illustrated in FIG. 11.

FIG. 13 is a rear view of an individual wearing the garment illustrated in FIG. 11 in an alternative position.

FIG. 14 is a front view of an individual wearing a garment according to an example embodiment of the present invention.

FIG. 15 is a top view of a garment of an exemplary embodiment of the present invention.

FIG. 16 is a top view of a multi-purpose garment according to an example embodiment of the present invention.

### DETAILED DESCRIPTION

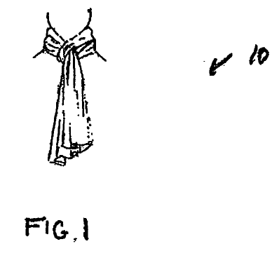

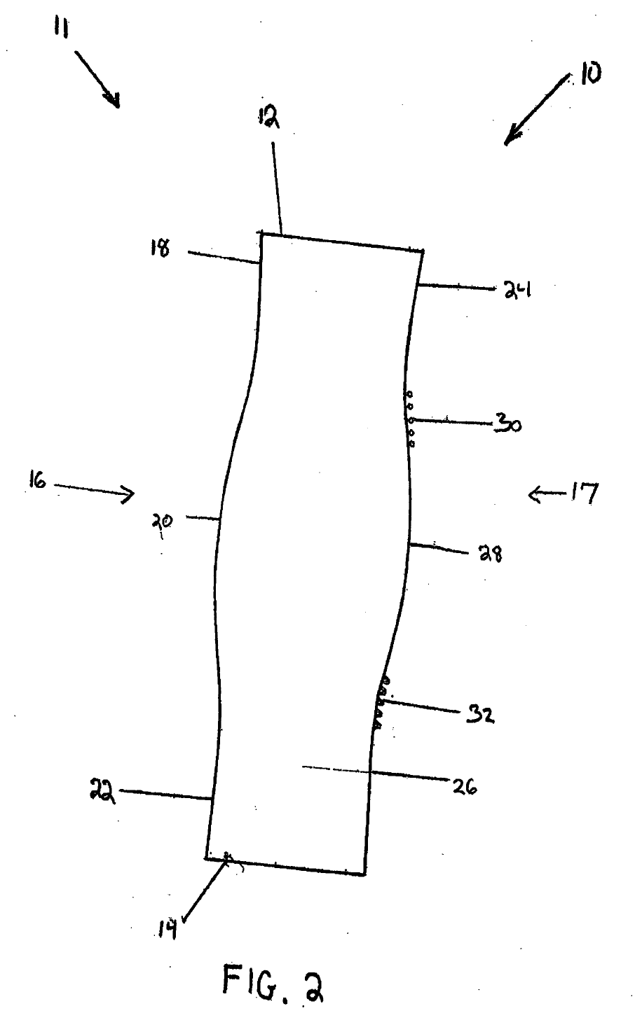

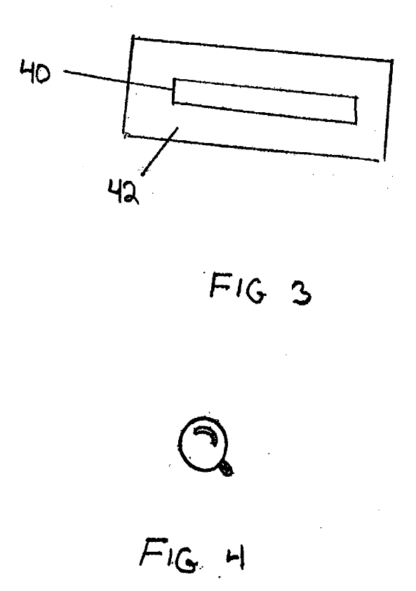

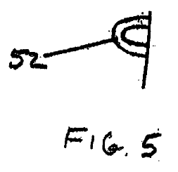

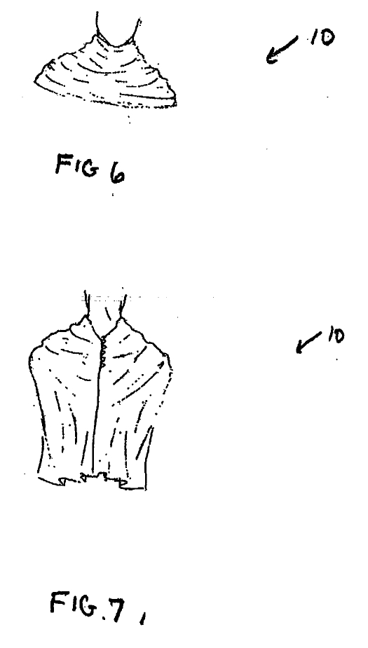

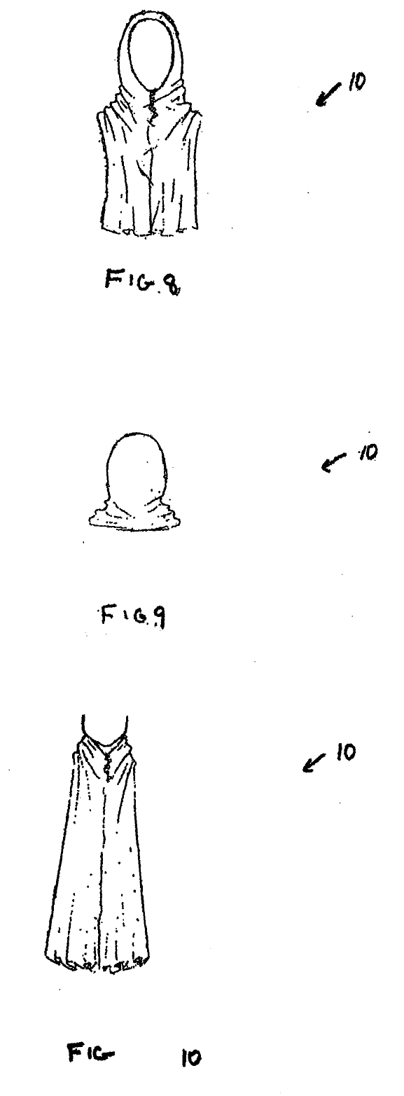

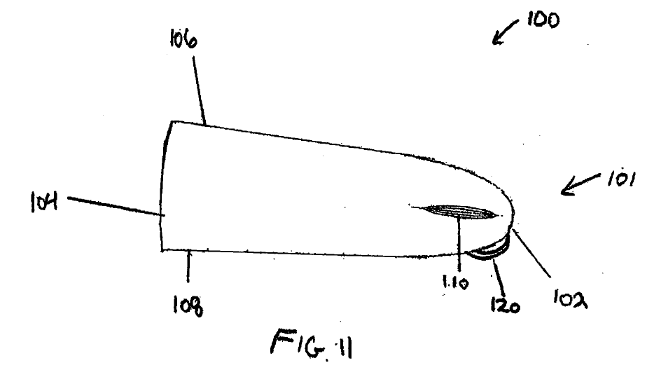

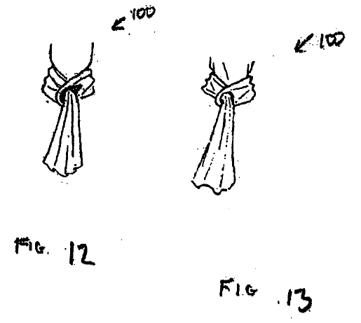

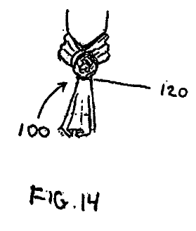

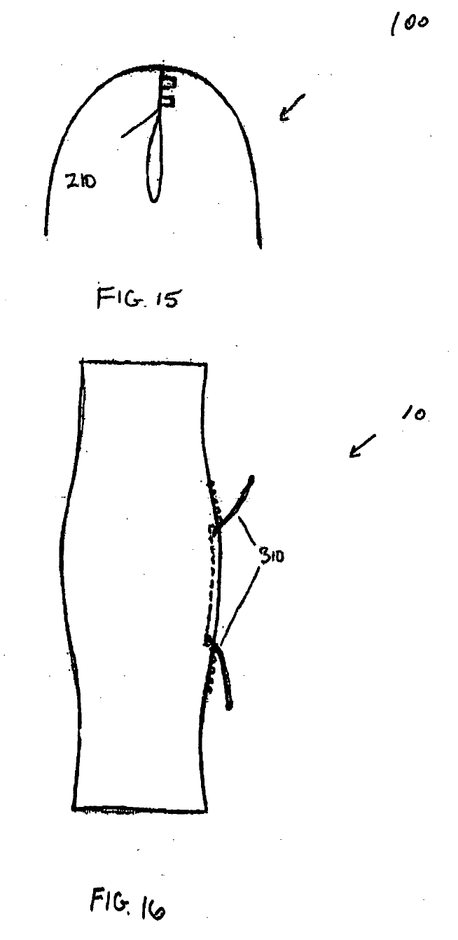

FIG. 1 illustrates a multi-purpose garment 10 of an example embodiment of the present invention as worn by an individual. The multi-purpose garment 10 may be worn by an individual for both protection from the elements as well as a fashion accessory.

FIG. 2 is a top view of the multi-purpose garment 10. The multi-purpose garment 10 is made from a body of material 11. The body of material 11 may be made of any material, e.g., wool, other woven or non-woven fabric, water repellant fabric, nylon, plastic, fur, rayon, silk, etc. The material may be selected to provide a comfortable feel against the skin of an individual or to limit static, bunching and wrinkles if worn exterior to other garments. The body of material 11 may have a composite construction, as illustrated in FIG. 3. As illustrated in FIG. 3, the body of material 11 may include an inner core 40 of waterproof material and an exterior non-waterproof material 42 surrounding the inner core 40. It should be appreciated that the layer of material 11 may include multiple layers of exterior material and/or multiple layers of core material.

Referring again to FIG. 2, the multi-purpose garment 10 has a first end 12, a second end 14, a first side edge 16 and a second side edge 17. The overall length of the multi-purpose garment 10 between the first end 12 and the second end 14 may be, e.g., approximately 60 inches. The narrowest width between the first side edge 16 and the second side edge 17 may be, e.g., 15.5 inches, and the widest width between the first side edge 16 and the second side edge 17 may be, e.g., 18.5 inches.

The first side edge 16, may have a complex shape including, e.g., first portion 18, a second portion 22 and a convex section 20 arranged between and connecting to the first portion 18 and the second portion 22. The first portion 18 abuts the first end 12 at a corner or vertex, and the second portion 22 abuts the second end 14 at a second corner or vertex. The convex section 20 may be rounded, parabolic or other non-linear shape, etc., that extends outwardly. The first portion 18 and/or the second portion 22 may be straight, concave, etc.

The second side edge 17 includes a third portion 24 that abuts the first end 12 at a third corner or vertex and a fourth portion 26 that abuts the second end 14 at a fourth corner or vertex. A convex section 28 is arranged between and connects the third portion 24 and the fourth portion 26. The convex section 20 and the convex section 28 are sized and configured such that when the multi-purpose garment 10 is worn by an individual, the widest width between the convex section 20 and the convex section 28 allow an individual's head, for example, to be covered.

A first section of a closure arrangement 30 is positioned on the second side edge 17. A second section of the closure arrangement 32 is also positioned on the second side edge 17. The first section of the closure arrangement 30 is illustrated as five buttons fastened to the second side edge 17. Although illustrated as, e.g., five buttons, the closure arrangement may include, e.g., any number of buttons, or alternative hook or loop ends, T-bar connectors, hook and eye, zipper, snaps, woven strands, etc. The second section of the closure arrangement 32 located on the second side edge 17, includes loops. FIG. 2 illustrates five loops positioned on the second side edge 17. The second section of the closure arrangement 32 mate with the first section of the closure arrangement 30 to provide a completed closure. The joining of the first section of the closure arrangement 30 and the second section of the closure arrangement 32 allows the multi-purpose garment 10 to be looped or connected to itself, allowing the user to be protected from the elements. The first section of the closure arrangement 30 and the second section of the closure arrangement 32 may be made of any appropriate materials, e.g., to withstand expected loading placed upon the multi-purpose garment 10. The closure arrangement may be configured as a “break away” connection, in which if a predetermined amount of load is placed upon the closure arrangement, the closure arrangement will disengage, thereby preventing e.g., strangulation. The breakaway force may be, for example, five pounds.

As illustrated in FIG. 4, the first section of closure arrangement may include a ball 50 that engages a loop 52 of the second section of the closure arrangement 32, which is illustrated in FIG. 5. Individual pairs of balls 50 and loops 52 may be positioned approximately 1.25 inches apart.

The multi-purpose garment may be worn in a variety of positions. As illustrated in FIGS. 6 and 7, the multi-purpose garment 10 may be wrapped around the neck with the closure arrangement engaged to protect the neck and shoulders of the individual, leaving the head exposed. While FIG. 6 is a front view and FIG. 7 is a rear view, it should be appreciated that the multi-purpose garment 10 may be worn in a reversed position, e.g., with the first end 12 and the second end 14 located in the front.

As illustrated in FIGS. 8 and 9, the multi-purpose garment 10 may be worn over the head to protect an individual from the elements, alleviating the need for a hat, while protecting the neck and front torso.

As illustrated in FIG. 10, the multi-purpose garment 10 may be worn around the neck draped over either the back or over the chest but not draped over the shoulders.

FIGS. 11 to 14 illustrate a garment of an example embodiment of the present invention.

The garment 100 includes a first distal end 102 and a second distal end 104. The first distal end 102 is connected to the second distal end 104 by a first side portion of 106 and a second side portion 108. The garment 100 is made of a body of material 101, which may be similar to that described above. Near the first distal end 102, additional material may be placed within the body of material 101 to stiffen the first distal end 102 to prevent bending in this area, if desired. The amount of material placed within the body of material may extend from the first distal end 102 to a distance corresponding to a farthest point of the opening 110 from the first distal end 102.

The second distal end 104 is illustrated as a rounded end. It should be appreciated, however, that other shapes or profiles are possible. The garment 100 may be reversible. An exterior material support arrangement 120 may be provided to receive and secure the second distal end 104 when worn.

FIGS. 12 and 13 illustrate the garment 100 as worn by an individual. The garment 100 is illustrated with the second distal end 104 inserted through the opening 110. After insertion of the second distal end 104 through the opening 110, the second distal end 104 may be tightened as desired, around the neck. As illustrated, the garment 100 may protect the neck and the chest. FIG. 12 illustrates the garment 100 worn on the front, and FIG. 13 illustrates the garment 100 worn on the back.

The opening 110 may be formed as a line or slit. Alternatively, opening 110 may also be circular or other desired geometric shape.

As illustrated in FIG. 14, the exterior material support arrangement 120 may be used to allow the material of the garment 100 to be supported on the front such that the material gathers around the neck.

As illustrated in FIG. 15, the garment 100 may have an opening arrangement 210, which may minimize or eliminate the possibility of strangulation. The opening arrangement 210 is made of hook-and-loop material or quick release snaps, etc that may open if excessive force is applied the arrangement 210.

As illustrated in FIG. 16. the multi-purpose garment 10 may include a drawstring 310 which is used for closure of the garment. The drawstring 310 is attached to the multi-purpose garment 10 to allow the multi-purpose garment 10 to be pulled snugly over the head, for example, providing a tight fit.

The garments described above may provide significant advantages over conventional scarves or shawls. For example, the multi-purpose garment 10 may be connected to itself to allow the garment to be worn securely. The multi-purpose garment may also allow selective sections of the body to be covered to allow selective protection of certain body parts.

The fabric of the body of material 11, 101 may have any desired pile density. The fabric may be water absorbent, e.g., to remove perspiration. The garment 10, 100 may be soaked in water and worn in any of the illustrated positions to cool the body parts in contact with the garment. A dense pile structure may be used to absorb moisture, whereas less dense pile may be used if absorbency is not necessary or desired or if an enhanced drape of the fabric is desired.

By using a high pile density, the hand or feel of the fabric may closely resemble that of a towel, for example. Decreasing the density of the fabric may reduce stability of the multi-purpose garment. The garment 10, 100 may be used to protect the user from the elements, such as if it is windy or cold. In hot weather, the garment 10, 100 may be used to cool a person, while the garment 10, 100 may also be used in the fall or winter time to provide warmth.

The garment 10, 100 may allow an individual to accessorize an outfit and provide a visually striking and pleasant appearance. The garment 10, 100 may be made of fur, knitted material, crocheted material, woven material, non-woven material, spun material, etc., and the exterior of the garment may be embroidered, decorated with beads, color-matched to outfits, etc.

What is claimed is:

1. A garment, comprising:
a material body with a first end, a second end, a first side edge and a second side edge;
a first section of a closure attached to the second side edge; and
a second section of the closure attached to the second side edge, the first section of the closure releasably connectible with the second section of the closure;
wherein the first side edge includes a first portion abutting the first end, a second portion abutting the second end, and a first convex section arranged between and connecting the first portion and the second portion, the second side edge including a third portion abutting the first end, a fourth portion abutting the second end, and a second convex section arranged between and connecting the third portion and the fourth portion.

2. The garment according to claim 1, wherein the first section of the closure includes at least one button.

3. The garment according to claim 1, wherein the second section of the closure includes at least one loop.

4. The garment according to claim 1, wherein the first section of the closure includes at least one button and the second section of the closure includes at least one loop.

5. The garment according to claim 1, wherein the first section of the closure arrangement includes five buttons.

6. The garment according to claim 1, wherein the second section of the closure includes five loops.

7. The garment according to claim 1, wherein the first section of the closure includes five buttons and the second section of the closure includes five loops.

8. The garment according to claim 1, wherein the first convex section is parabolic.

9. The garment according to claim 1, wherein the second convex section is parabolic.

10. The garment according to claim 1, wherein the first and second convex sections are parabolic

11. The garment according to claim 1, wherein the first portion, the second portion, the third portion and the fourth portion are one of (a) straight and (b) concave.

12. The garment according to claim 1, wherein a material of the body of material is waterproof.

13. The garment according to claim 1, wherein the body of material includes an inner core of waterproof material and an exterior of non-waterproof material surrounding the inner core.

14. The garment according to claim 1, wherein the body of material is sized to be selectively worn as a scarf, a shawl and a wrap.

15. A garment comprising:
a body of material including a first distal end, a second distal end, two opposing side portions connecting the first distal end and the second distal end, and an opening located along a longitudinal axis of the body of material extending between the first distal end and the second distal end, the opening arranged to between the first distal end and the second distal end;
a layer of reinforcing material placed within the body of material extending longitudinally from the first distal end toward the second distal end at least to a location corresponding to a farthest distance of the opening from the first distal end; and
an exterior material support arrangement connected at least to one side portion, the body of material wrappable around a neck of a wearer with the second distal end passed through the opening and secured by the exterior material support arrangement.

16. The multi-purpose garment according to claim 15, wherein the opening is circular.

17. The multi-purpose garment according to claim 15, wherein the opening has a shape of one of a line, a circle, a square, a star, an octagon, a triangle, a pentagon, a hexagon and a crescent.
